(19) United States
(12) Patent Application Publication
(10) Pub. No.: US 2006/0009803 A1
(43) Pub. Date: Jan. 12, 2006

(54) Suture device for soft tissue and/or fixation of soft tissue to bone

(76) Inventor: Alberto Luis Garay, Buenos Aires (AR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/925,315

(22) Filed: Aug. 24, 2004

(30) Foreign Application Priority Data
Sep. 4, 2003 (AR) PO3 01 03205

(51) Int. Cl. A61B 17/08

(52) U.S. Cl. 606/217

(57) ABSTRACT

A suture device for soft tissue which comprises at least a zipper band, wherein at least one of the ends thereof cooperates with at least one zipper lock which is not fixed to said band is disclosed. Also disclosed is a device for the fixation of soft tissue to bone. The object of this invention is to develop a suture process for soft tissue and/or fixation of soft tissue to bone based on the utilization of zipper bands, preferably made of flexible plastic material, which cooperate with zipper locks which are not fixed to said bands.

### FIELD OF INVENTION

The present invention relates to a suture device for soft tissue and/or fixation of soft tissue to bone. More specifically, it refers to a suture device for soft tissue and/or fixation of soft tissue to bone which enables the necessary compression to be attained, regardless of the surgeon's ability and with fewer elements, thus representing significant savings in terms of cost and operative times.

### BACKGROUND

U.S. Pat. No. 3,570,497 discloses a suture element comprising a needle on one of the ends thereof, a band bearing a zipper and a lock for said band. The stop is provided with an opening for the band zipper to pass therethrough, so that when passed through by said needle, the band passes through said opening until a suture is completed on the tissue surface, it being maintained in place by the zipper, and being much faster than the conventional ones with stitch knots.

On the other hand, U.S. Pat. No. 5,879,371 discloses a fixation means for soft tissue anastomosis via surgery (or endoscopy) which represents a minimal trauma. Fixation means comprises a needle, a suture and a splint. The needle and the suture means enable stitching of the fixation means through, for instance, a graft and an artery, in order to join them. The splint allows the formation of a loop without knotting the suture element.

U.S. Pat. Nos. 5,993,476 and 5,449,359 disclose an osteosynthesis clamp. Said clamp comprises a “U” body with two spaced legs, each leg comprising a distal end which can be inserted into a bone opening. Proximate ends of said at least two legs are connected to the ends of a deformable bridge comprising two sections which are separated one from the other and exhibit a space therebetween, same being force-deformable.

U.S. Pat. No. 5,242,457 discloses a surgical clamp intended for human tissue suture which comprises a body made of deformable material and includes a loop through which the suture thread passes.

### SUMMARY

In all of the above cases, the object has been to simplify soft tissue and/or soft tissue to bone fixation suture, regardless of the surgeon's ability. Nevertheless, said sutures and/or soft tissue fixation stitches may be loose depending on the surgeon's ability and/or accessability of the intervened area, whereby the operation has to be repeated with a new fixation element and a new invasion of the tissues involved, thus increasing the traumatic effect suffered by the patient.

There follows a detail on other devices for the fixation of soft tissue (joint capsules, tendons, ligaments) to bone. Said soft tissue must be fixed to bone via a sufficiently stable device in order to allow rehabilitation therapy to start as soon as possible. This is already so in knee surgery, in which new fixation devices for cruciate ligaments are very strong, and thence immobilization by means of plaster casts is not required. At present soft tissue fixation devices are the following:
1) interference screws: ligament or tendon is introduced into a hole made on the bone and a screw is applied into said hole. Threads fix tendon against the hole wall. Drawback thereof is that when the screw is turned the tendon also turns and fibers thereof may be cut should the adjustment be too tight;
2) screws with spiked washers: screw tightening guides washer spikes towards and through tendon fibers, pressing same against the bone. Drawback thereof is that tendon is unevenly pressed, thereby producing inclination of said washer; and
3) anchor: a device bearing two projections on one of its ends and two sutures for tying the tendon on the other end, this being the weak point of this device.

Although some of the devices have produced good results, surgeons still find that some connection elements are bulky and complicated on one hand; and on the other hand some of the smaller elements, such as clamps, are unable to attain an efficient fixation amidst bones, amidst tissues, or between tissues and bones.

Accordingly, one of the problems to be solved is to attain the necessary permanent compression of tissues to be repaired and/or fixed to bones, with as little regards as possible to the surgeon's ability.

As it is indispensable to work through the patient's wound for contacting the different parts of the fixation elements and further fixation thereof, it is preferable that structure and fixation of said fixation elements be as simple as possible, so as to reduce intervention times and the consequent trauma.

This invention solves the above problems by means of a suture device for soft tissues and/or fixation of soft tissues to bones which is based on the use of zipper bands and separate zipper locks, i.e. not fixed to said zipper bands. Further, such zipper locks may form part of a “U” shaped clamp structure for the fixation of soft tissue to bone. In all of the cases a sharp element associated to one of the bands end must be used in order to carry out suture. More specifically, said sharp element is a suture needle which may have different configurations according to the suture type. On the other hand, zipper bands are preferably made of flexible plastics material and may be reabsorbable or not as the case may be.

Independence between said zipper locks and said zipper bands enables manufacturing of such bands in several lengths. Further, said bands may be commercialized in bulk, wound on reels, for the surgeon to cut lengths thereof according to the surgical needs.

As regards separate zipper bands, these may comprise on one of the ends thereof a stop element which will hold the first stitch. Upon the lack of said stop element, it is also possible to stitch said end of said zipper band onto said first stitch by means of an ordinary suture thread which shall obviously pass through holes made on such end of said zipper band. However, it is preferable to insert zipper locks on both ends of said band once it is passed through the wound lips, displacing same in opposite directions in order to attain the necessary compression between said lips.

This kind of suture may be done stitch by stitch by means of independent zipper bands, or continuously through the successive and alternate passing of a single zipper band through the lips of a wound. In this case zipper locks may be used both at the beginning and at the end of the suture; or else, each time said zipper band passes through a lip of the wound. The later arrangement provides a better compression control in the case of a wound of considerable size. As we have already mentioned, necessary compression is achieved by displacing each zipper locks pair which are faced in opposite directions.

Also, and depending on the case, such locks may be associated to a soft tissue or bone fixation element. In this case, such locks may be fixedly associated thereto or it may be necessary that same be able to rotate with regards to said associated fixation element, in order to enable orientation thereof as per surgical needs.

It is also possible for the suture needles to be sold separately from the zipper bands. Assembly of said suture needle to said zipper band is done between one of the ends of said needle, which end may be hollow or threaded, and one of the ends of said bands. More specifically, should the needle exhibit a hollow end, the end of the band is inserted into said hollow end and subsequently it is pressure-deformed by means of an adequate tool, in order for the end of the band to be secured in the interior of said hollow end.

On the other hand, should the needle end be threaded, the end of said band will have a thread complementing that of the end of said needle.

It is then an object of the present invention a suture device for soft tissues which comprises at least a zipper band, wherein at least one of the ends thereof cooperates with at least one zipper lock which is not fixed to said band

Still another object of the present invention is a device for the fixation of soft tissue to bone, which comprises at least one bone fixation element which includes at least a bone fixation element which includes at least one zipper lock which cooperates with one of the ends of at least one zipper band which is not fixed to said fixation element.

In order to implement the above briefly discussed advantages and facilitate understanding of constructive and functional characteristics of the present invention, there follows a description of preferred exemplary embodiments, which are schematically illustrated without a determined scale by the following detailed figures.

### BRIEF DESCRIPTION OF THE DRAWINGS

FIG. 1 depicts an assembly of zipper band passing through the lips of a wound stitch by stitch associated to zipper locks in order to attain the necessary compression.

FIG. 2 depicts a zipper band passing through the lips of a wound in alternate and continuous manner.

FIG. 3 is a top view of a zipper band which is associated to a sharp element on one of its ends and to a stop on the other.

FIGS. 4A and 4B depict different zipper locks configurations.

FIG. 5A depicts a “U” shaped bone fixation element with serrated legs which include two inverted zipper locks, through which there pass each of the ends of a zipper band.

FIG. 5B depicts a “U” shaped bone fixation element with serrated legs which includes a single zipper lock, through which there crosses one of the ends of a zipper band.

FIG. 6A depicts a bone fixation element via a harpoon or screw which includes two mutually inverted zipper locks.

FIG. 6B depicts a bone fixation element via a harpoon or screw which includes a single zipper lock.

FIG. 7A depicts a bone fixation element via a fastening screw which includes two mutually inverted zipper locks.

FIG. 7B depicts a bone fixation element via a fastening screw which includes a single zipper lock.

FIG. 8A depicts a soft tissue fixation element via a safety pin which includes two mutually inverted zipper locks.

FIG. 8A depicts a soft tissue fixation element via a safety pin which includes a single zipper lock.

FIG. 9 depicts the use of the device of the invention on a kneecap fracture.

FIG. 10 depicts a bone fracture requiring a steel plate and using the device of the invention.

### DETAILED DESCRIPTION OF THE PREFERRED EMBODIMENTS

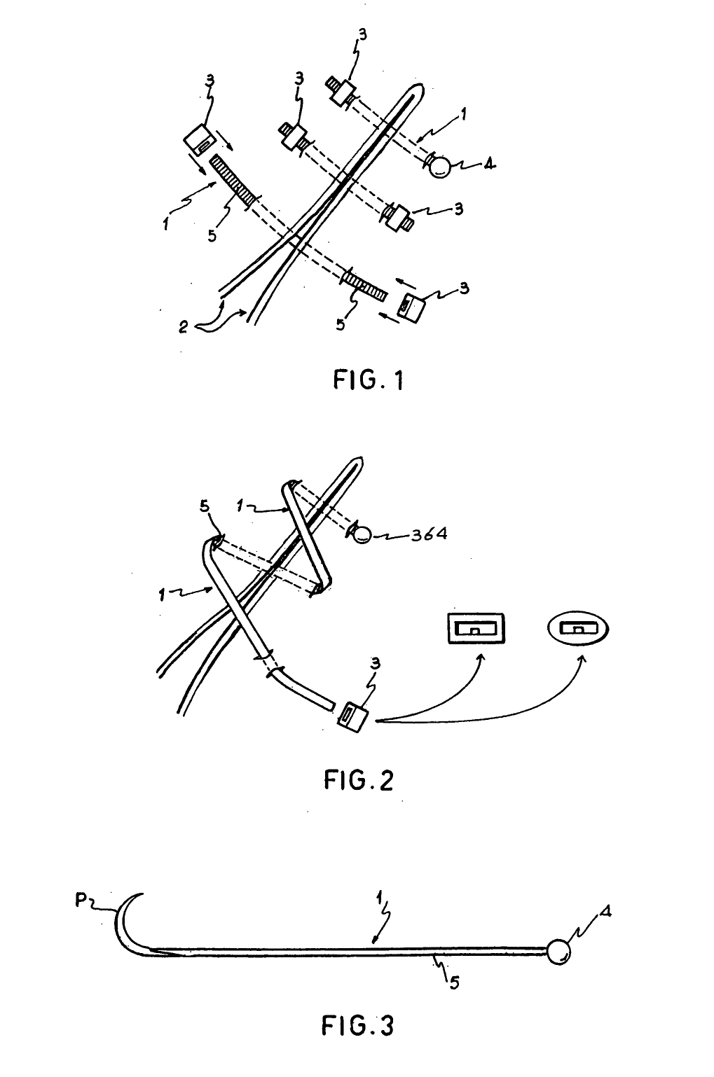

Said FIG. 1 shows three zipper bands 1 passing through the lips of a wound 2, through which ends there are inserted zipper locks 3. In order to close the wound zipper locks 3 are displaced in mutually opposite senses, the ends of said zipper bands 1 being held while said displacement is produced and until the required compression is reached.

A variant of the above case shows that the end of a zipper band 1 passing through the first stitch may include a stop element 4 instead of a zipper lock 3, in order to establish an anchor stitch or initial stitch. Said fixation of said end of said band 1 to said stitch may also be done with an ordinary suture thread between said band 1 and said wound 2. Said anchor stitch or initial stitch may also be obtained by means of the association of said zipper lock 3 to an element for the fixation to soft tissue 6, for instance a safety pin 15, as discussed below.

FIG. 2 shows a suture alternative exhibiting a single zipper band 1. In this case a zipper lock 3 is inserted in each of the ends of the zipper band 1, once this has been threaded through the lips of the wound 2. As previously mentioned, zipper lock 3 of the end of zipper band 1 may be replaced at the first stitch by a stop element 4 or an ordinary suture thread, in order to establish an anchor stitch or initial stitch.

Should a higher control of the suture be desired, the end of the band which continuously and alternately passes through the lips of wound 2 may also pass through successive zipper locks 3, namely locks 3 which are mutually faced at each performed stitch.

It is to be noted that in certain cases it could prove necessary for band 1 to possess zippers on both faces.

Once the desired compression is achieved, the remainder of said zipper band 1 is cut.

In all of the cases, in order to pass through wound 2 with one or more zipper bands 1, a sharp element (P) will be needed as guide, such as for instance a suture needle, associated to one of the ends of such band 1, as can be seen in FIG. 3. Suture needle may exhibit different shapes and sizes, as follows:
straight and half curve, in both cases susceptible of being triangular or round; and
curve, this also being susceptible of being triangular or round.

Needle tip may be of different shapes as well as the edges of the needle, and the manufacturing material thereof may be tempered stainless steel or the like.

Elements conforming the suture devices of the invention may be of reabsorbable material, as for example polydioxanone, or non-reabsorbable material, such as silastic, silicones, polyethylene, polyester, nylon, metals, etc. Sizes may be comprised within varied ranges as well as shapes and thickness thereof, according to the requirements and characteristics of the tissues with which they are to be used. Particularly, zipper bands 1 are flexible.

Zippers 5 of bands 1 profiles may be rectangular, round, square, semi-round, triangular or the like and same may be on one or both faces of said bands 1. In turn, said bands may be sold in bulk, as for example as a single band continuously wound on a reel so that the surgeon may cut lengths thereof as required.

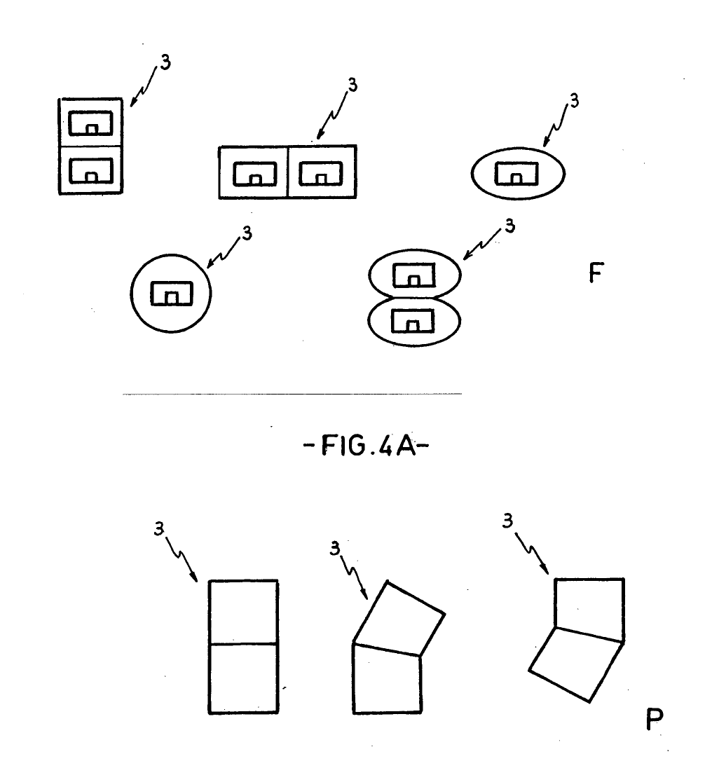

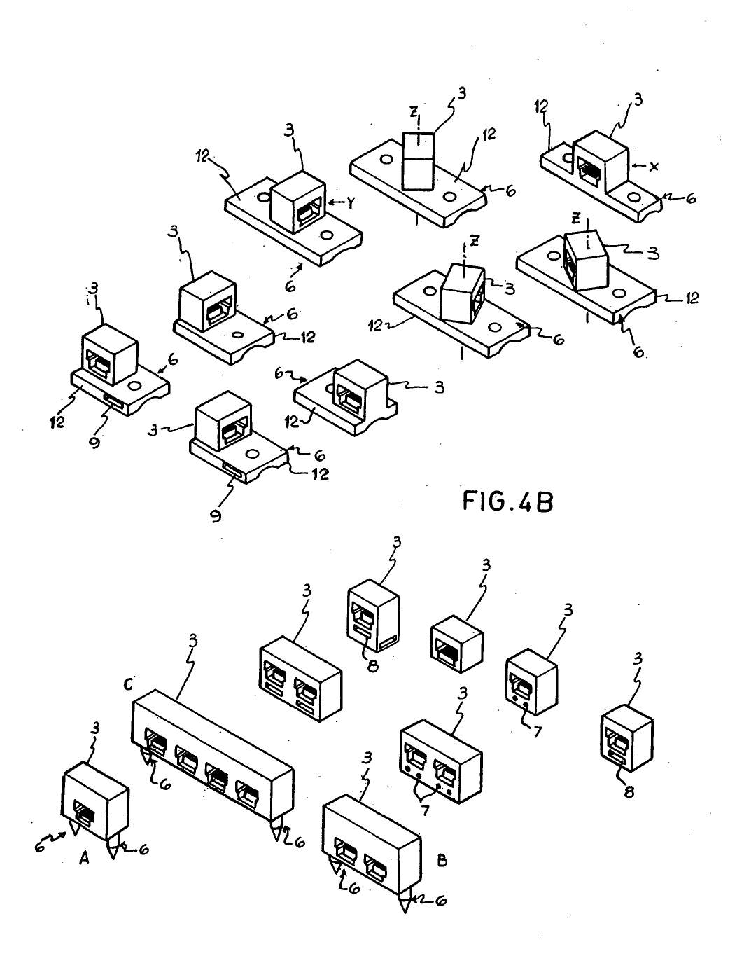

FIG. 4A depicts different zipper locks 3, which may be rectangular, ovoid, spherical, or the like. However, a blunt form is preferred in order to avoid damaging tissues. Further, said locks 3 may associate to form an assembly of several locks 3, for example two. In the later case, one over the other, one over the other and constituting an acute angle, one beside the other, one beside the other and constituting an acute angle, and one beside the other forming an obtuse angle.

Said locks 3 may be associated to the structure of different fixation elements such as “U” shaped plates or clamps 10 which are fixed to bone parts, or base plates 12 which are fixed to the bone by means of fixation screws, harpoons, wires or the like, as will be seen below.

FIG. 4B depicts different structures of zipper locks 3. Said locks 3, as previously mentioned, may be associated in order to form an assembly of one or several locks 3, which may comprise bones or soft tissue fixation elements 6, holes 7 for the ordinary suture thread passage and/or loops 8 for another zipper band 1 passage. All of the zipper locks 3 may exhibit the same insertion direction for bands 1. However, a configuration of alternate inverted locks 3 as also shown by said FIG. 4B is particularly useful when a zipper band passes through a soft tissue as for example a joint capsule, forming a loop. Consequently, zipper 5 on the ends of said band 1 which will pass through said locks 3 will be inverted, whereby these shall also be inverted in order to cooperate with said zipper 5.

Another embodiment provides for said locks 3 to rotate around their vertical axis (Z) with respect to the fixation element 6 to which same are associated. Also contemplated is the option of including guides 9 on the base thereof in order to allow for a fine adjustment when approaching wound lips 2.

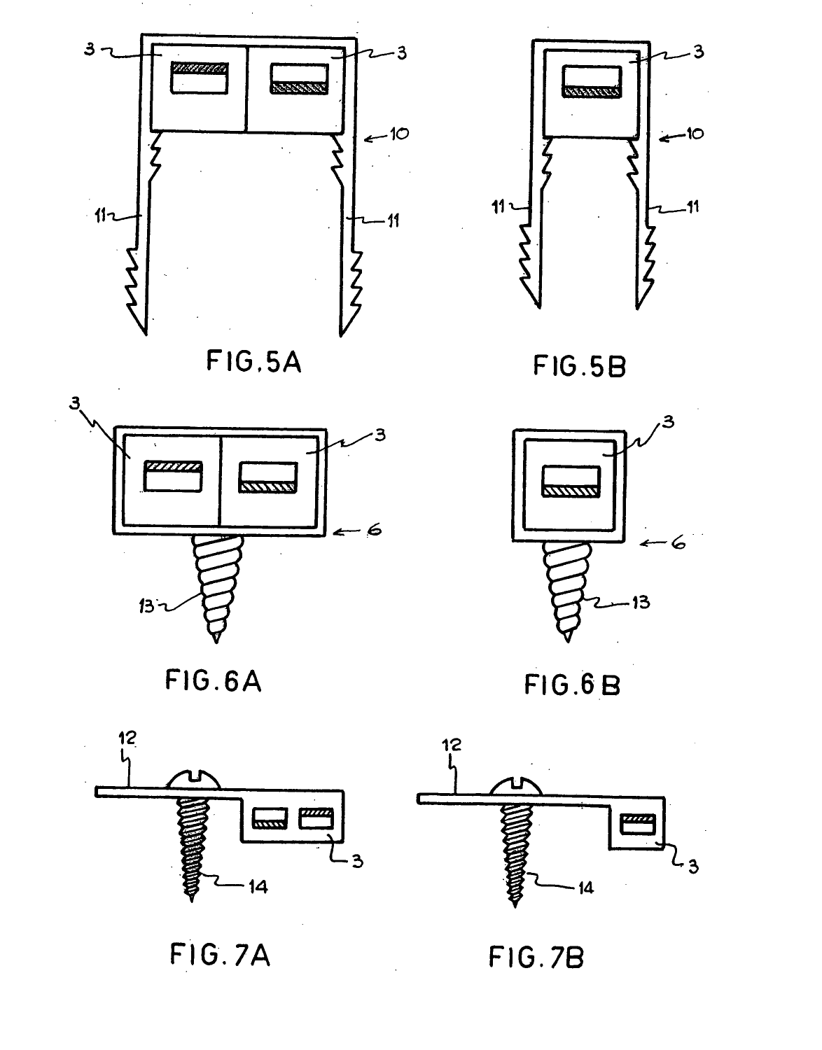

FIGS. 5A and B depict zipper locks 3 included in the structure of a “U” shaped clamp 10 with serrated legs 11, particularly applicable to the fixation of soft tissue, as for example joint capsules, tendons or ligaments, to bones; or to joints movements limitation. All of the above, which relates to different configurations and types of zipper locks 3 is also valid for the present embodiments. In the particular non-limitative case depicted by FIG. 5A there can be seen two zipper locks 3 which are mutually inverted on the basis of the above. However said “U” shaped clamp 10 may also include a single zipper lock 3, as illustrated by FIG. 5B.

FIG. 6A depicts another embodiment wherein said bone fixation element 6 consists of two mutually inverted zipper locks 3, which are fixed to the bone by means of harpoon 13. FIG. 6B is a variant of FIG. 6A and includes a single zipper lock 3.

FIG. 7A depicts a new variant of an assembly of two mutually inverted zipper locks 3, included in a base plate 12 which is fixed to bone by means of a fixation screw 14. FIG. 7B depicts a base plate 12 which includes a single zipper lock 3.

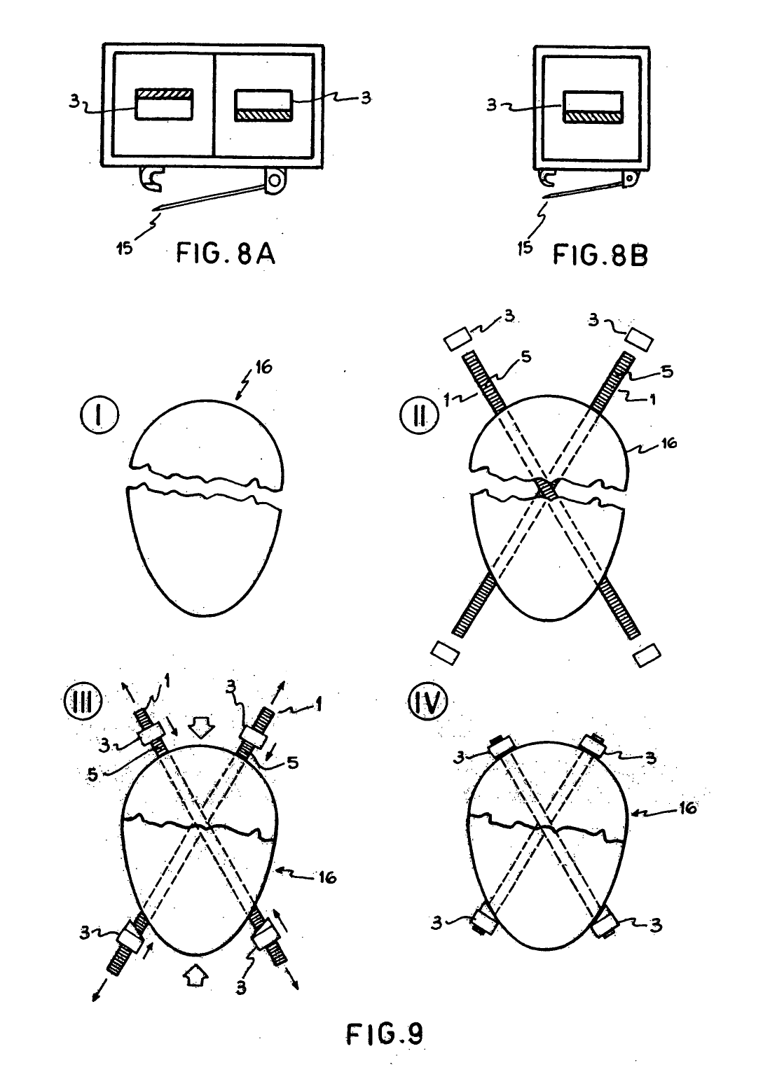

FIG. 8A illustrates a last variable, already mentioned for the three previous cases, which contemplates an assembly of two mutually inverted zipper locks 3, which are to be fixed to a soft tissue by means of a safety pin 15. FIG. 8B contemplates the case of a single zipper lock 3. In all of the cases described by FIGS. 5B through 8B, which contemplate that one of the ends of a zipper band 1 pass through a single zipper lock 3, it will be necessary for the other end of said zipper band 1 to include a stop element 4 or to pass through another zipper lock 3, or to be fixed to soft tissue by means of a suture thread.

The process wherein a soft tissue is fixed to a bone is actually simple. The fixation element 6 is fixed to the bone which includes at least a zipper lock on the one side, and a zipper band 1 is passed through the soft tissue to be fixed to said bone, on the other, using a sharp element (P) as a guide, as has been previously mentioned. Once said soft tissue is passed through by said zipper band 3, sharp element (P) is released and one of the ends of said band 1 is inserted into at least one zipper lock 3 included in said bone fixation element 6, as for example a “U” shaped clamp. The other end of said band 1 will pass through another zipper lock 3 included in said bone fixation element 6 or else, as has been mentioned above, same will include a stop element 4 or be inserted into another zipper lock 3 or stitched to soft tissue by means of a suture thread. Lastly, surgeon will pull at least one end of said zipper band 1 until the desired fixation is attained.

This type of suture device, which novelty resides in the independence between zipper bands 1 and zipper locks 3, may be applied to a large variety of surgical fields, particularly traumatological ones. For example, FIG. 9 in its stages I-IV illustrates a kneecap fracture 16 in which the device of the invention is used. Zipper bands 1 are passed through holes made on said bone by means of an electric drill and then zipper locks 3 are inserted into the ends. By displacing the latter towards said bone the necessary compression for the bone callus to be formed is attained.

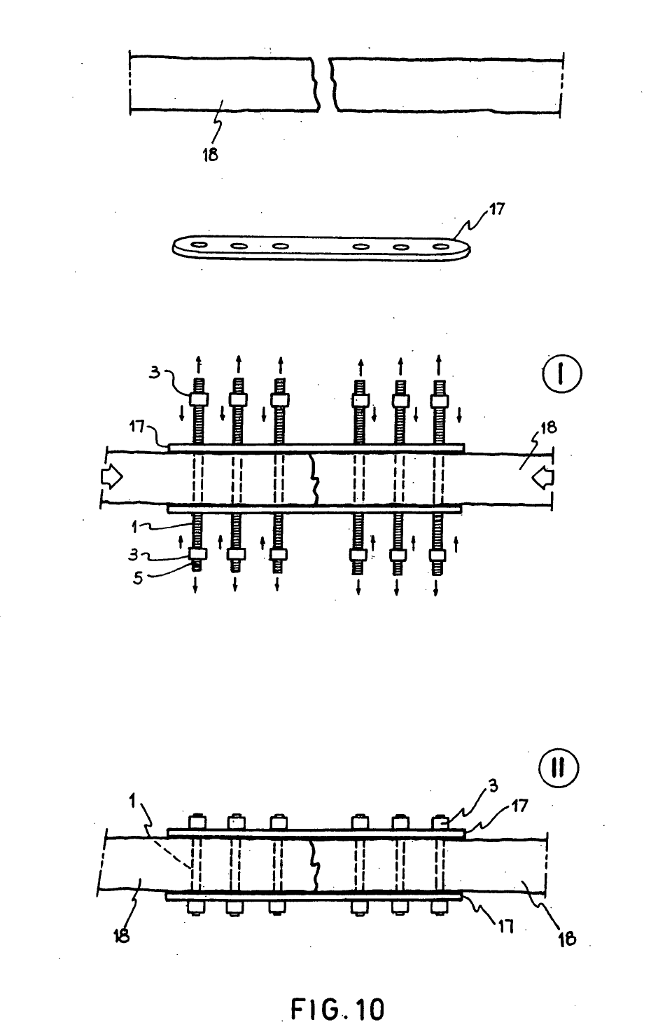

On the other hand, FIG. 10 illustrates in its stages I and II an application of the present device for another type of fracture which requires use of a steel plate 17, which plate is fixed to the broken bone contour 18 by means of the combination of zipper bands 1 and zipper locks 3 of the present invention. Bands 1 are passed through holes made into the broken bone by means of an electric drill, ends of said bands 1 are inserted through holes of steel plates 17 and lastly zipper locks 3 are inserted so that when displaced towards the broken bone they may attain the necessary compression between said plate 17 and said bone 18.

What is claimed is:

1. Suture device for soft tissue, characterized in that the device comprises at least one zipper band having first and second ends, wherein the first end cooperates with at least one zipper lock which is not fixed to said band.

2. A device according to claim 1, characterized in that the second end of said at least one zipper band either comprises a stop element, cooperates with another zipper lock or is sutured to a soft tissue by means of a suture thread.

3. A device according to claim 1, wherein said at least one zipper lock is made double and joined to one another either side by side, on top of one another, next to each other at an acute angle, or next to each other at an obtuse angle.

4. A device according to claim 3, characterized in that said zipper locks further include holes for suture threads to pass therethrough, loops for a zipper band to pass therethrough, guides for a fine adjustment of an approach thereof onto lips of a wound and/or soft tissue fixation elements, as for example a safety pin.

5. A device for the fixation of soft tissue to bone, characterized in that the device comprises at least one bone fixation element comprising at least one zipper lock which cooperates with one of the ends of at least one zipper band not fixed to said fixation element.

6. A device according to claim 5, characterized in that said at least one bone fixation element is a “U” shaped clamp with legs.

7. A device according to claim 6, characterized in that said legs include teeth or exhibit a serrated surface.

8. A device according to claim 5, characterized in that said fixation element comprises a base plate with at least one zipper lock associated thereto and which is fixed to the bone by means of fixation screws, harpoons and the like.

9. A device according to claims 1 or 5, characterized in that one of the ends of said at least one zipper band is associated to a sharp element.

10. A device according to claim 9, characterized in that said sharp element is a suture needle.

11. A device according to claim 10, characterized in that said suture needle is associated to one of the ends of said at least one zipper band by means of a thread.

12. A device according to claim 10, characterized in that a non-sharp end of said suture needle is hollow and able to receive an end of said at least one zipper band, in order to be then pressure-adjusted thereto.

13. A device according to claim 10, characterized in that said suture needle may be straight or half curve, and may have a triangular or circular cross section.

14. A device according to claim 10, characterized in that a tip and edges of said suture needle posses different shapes, said needle being made of tempered stainless steel or the like.

15. A device according to claims 1 or 5, characterized in that said zipper has a cross-section that is rectangular, round, quadrangular, semi-round, triangular or the like.

16. A device according to claim 5, characterized in that said at least one zipper lock is able to rotate around a vertical axis (Z) with respect to the fixation element to which the zipper lock is associated, in order to be oriented as per surgical needs.

17. A device according to claims 1 or 5, characterized in that said at least one zipper lock is preferably blunt and has a rectangular, ovoid, or spherical shape.

18. A device according to claims 1 or 5, characterized in that the elements conforming said device are made from re-absorbable materials, as for example polydioxanone, or from non-reabsorbable materials such as silastic, silicones, polyethylene, polyester, nylon or metals.

19. A device according to claims 1 or 5, characterized in that said at least one zipper band is flexible.

20. A device according to of claims 1 or 5, characterized in that said at least one band bears a zipper on both faces.
